(19) United States
(12) Patent Application Publication
(10) Pub. No.: US 2022/0354455 A1
(43) Pub. Date: Nov. 10, 2022

(54) DEVICE FOR HOLDING A MEDICAL INSTRUMENT AND APPARATUS COMPRISING SUCH A DEVICE

(71) Applicant: KOELIS, Meylan (FR)

(72) Inventors: Antoine LEROY, Bois d' Arcy (FR); Tristan BRONSARD, Le Touvet (FR)

(73) Assignee: KOELIS, Meylan (FR)

(21) Appl. No.: 17/633,400

(22) PCT Filed: Aug. 6, 2020
(86) PCT No.: PCT/EP2020/072184

§ 371 Date: Feb. 7, 2022

(30) Foreign Application Priority Data
Aug. 8, 2019 (FR) 1909085

(51) Int. Cl. A61B 8/00; A61M 25/06

(52) U.S. Cl. CPC A61B 8/4209; A61M 25/065; A61N 2005/1012

(57) ABSTRACT

The invention relates to a holder device for holding a medical instrument, the device comprising:

a base that is to be carried by an apparatus comprising a medical imaging probe associated with the medical instrument; and

a carrier structure for carrying the medical instrument and movably mounted on the base so as to be capable of moving on the base between a first position and a second position.

The invention also relates to an installation including both such a device and a needle-guide.

The invention relates to a holder device for holding a medical instrument.

The invention also relates to an installation including such a device.

### BACKGROUND OF THE INVENTION

As a result both of progress in the technologies used and also of good monitoring of the population at risk, cancers of the prostate are being detected earlier and earlier. Consequently, the tumors that are found are getting smaller and smaller.

Conventional treatments of total or partial ablation of the prostate are thus nowadays no longer appropriate, in particular in the light of the severe post-operative consequences to which they give rise (incontinence, impotence, . . . ).

Thus, operators are turning increasingly to targeted treatments consist in inserting an instrument into the patient's body in order to bring the instrument close to a particular zone of the prostate for treatment. By way of example, the treatment may be brachytherapy with insertion of a catheter (for positioning radioactive seeds), or it may be other targeted therapies such as cryotherapy or laser therapy.

A prerequisite for treatment of this type is that the operator needs to be highly accurate in order to approach the target zone correctly with the instrument. However, the instrument sometimes needs to be held in place for several minutes to several tens of minutes so that the treatment can be performed properly, and this is uncomfortable for the operator. Also, it is difficult for the operator to control small involuntary movements of the hand due to the prolonged static position during the treatment. Unfortunately, these unwanted movements that can turn out to be dangerous for the patient, in particular when they lead to the instrument being pushed further into the prostate.

### OBJECT OF THE INVENTION

An object of the invention is to propose a holder device for holding a medical instrument that serves to limit any risk of involuntary movement of the instrument while it is being used to treat a particular targeted zone of a patient's body.

Another object of the invention is to provide an installation including such a device.

### SUMMARY OF THE INVENTION

In order to achieve this object, the invention provides a holder device for holding a medical instrument, the device comprising:

a base that is to be carried by an apparatus comprising a medical imaging probe associated with the medical instrument; and

a carrier structure for carrying the medical instrument and movably mounted on the base so as to be capable of moving on the base between a first position=and a second position.

The invention thus enables the medical instrument to be secured temporarily to the holder device in order to assist the work of an operator.

In particular, the invention enables the position of the medical instrument to be held stationary without action on the part of the operator. It should be observed in particular that if the instrument is pushed into the target organ, the invention enables the depth of the instrument in the target organ to be kept constant.

Nevertheless, should it be necessary for the medical instrument can be removed from the holder device, that can be done quickly.

In particular manner, the device is suitable for holding a medical instrument in the form of an applicator comprising a handle from which there extends a treatment needle, the carrier structure including, for this purpose, a ring for carrying the handle of the medical instrument.

In particular manner, the ring is not carried by a needle-carrier of the device.

In particular manner, the carrier structure is slidably mounted on the base.

In particular manner, the carrier structure comprises a column movably mounted on the base and a ring movably mounted on the column.

In particular manner, the ring is slidably mounted on the column.

In particular manner, the ring is pivotally mounted on the column.

In particular manner, the ring includes means for fastening the instrument temporarily to the device.

In particular manner, the ring includes at least one window for viewing the column.

In particular manner, the ring includes at least one position-marker symbol for identifying the orientation of the ring relative to the column.

In particular manner, the column includes at least one succession of graphical marks.

In particular manner, the carrier structure can be mounted on the base in at least two different engagement directions.

In particular manner, the carrier structure includes a direction-marker element for each possible direction for engaging the carrier structure on the base.

In particular manner, the base includes at least one position-marker sign for symbolizing where the base is to be mounted in service on the apparatus.

The invention also provides an installation comprising an above-described device and a needle-guide.

Optionally, the needle-guide is a grid.

Optionally, the installation also comprises an apparatus that includes an ultrasound probe.

Optionally, the base and/or the carrier structure is a part that has been injection molded.

Optionally, the installation includes the medical instrument that is not a needle-guide.

Other characteristics and advantages of the invention appear on reading the following description of particular, nonlimiting embodiments of the invention.

### BRIEF DESCRIPTION OF THE DRAWINGS

The invention can be better understood in the light of the following description given with reference to the accompanying figures, in which:

FIG. 1 shows an installation in a first embodiment of the invention;

FIG. 2 is substantially a face view of the device of the installation shown in FIG. 1, with a ring of the device being in a first position;

FIG. 3 is a view similar to the view of FIG. 2, with a ring of the device being in a second position;

FIG. 4a shows a first step in fastening the holder device to an apparatus of the installation shown in FIG. 1;

FIG. 4b shows a second step in fastening the holder device to an apparatus of the installation shown in FIG. 1;

FIG. 4c shows a third step in fastening the holder device to an apparatus of the installation shown in FIG. 1;

FIG. 4d shows a fourth step in fastening the holder device to an apparatus of the installation shown in FIG. 1;

FIG. 4e shows a fifth step in fastening the holder device to an apparatus of the installation shown in FIG. 1;

FIG. 4f shows a sixth step in fastening the holder device to an apparatus of the installation shown in FIG. 1;

FIG. 5 shows an installation in a second embodiment of the invention; and

FIG. 6 shows an installation in a third embodiment of the invention.

### DETAILED DESCRIPTION OF THE INVENTION

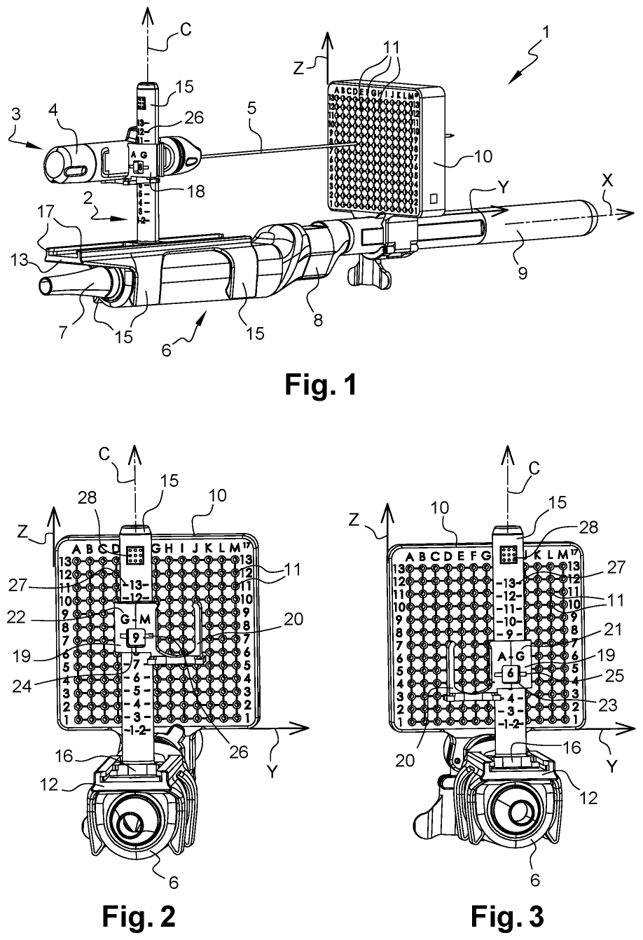

With reference to FIGS. 1 to 3, in a first embodiment of the invention, the installation, which is given overall reference 1, comprises a holder device 2 that is associated with a medical instrument. In this example, the medical instrument is an applicator 3 for applying microwave treatment to at least one zone of a patient's prostate and to do so by means of a transperineal approach.

Naturally, this application is not limiting, and, like the holder device 2, the installation 1 can be used in association with various different types of medical act (biopsy, brachytherapy, cryotherapy, laser therapy, . . . ); with various different approaches (transperineal, transrectal, . . . ); and with various different targets (prostate, kidney, liver, uterus, . . . ).

The installation 1 and the holder device 2 thus be associated with instruments other than the applicator 3, e.g. with a biopsy needle-carrier.

In order to be able to carry out targeted treatment, the applicator 3 includes in particular a handle 4 from which there extends a treatment needle 5. The handle 4 also includes a connector for attaching it to a sterile cable. Such an applicator 3 is well known to the person skilled in the art, and is not described in detail herein.

The installation 1 also includes a monitoring apparatus 6 that extends generally longitudinally along an axis Z in order to facilitate insertion into the patient's rectum.

Preferably, the apparatus 6 comprises a support 7 that extends parallel to the axis Z, that is to be gripped at its proximal end by an operator, and that is extended at its distal end by a crank 8 of the apparatus 2. Said crank 8 is itself extended by a medical imaging probe 9 of the apparatus 2 for generating three-dimensional images of the prostate and of its surroundings, which probe 9 also extends along the axis Z. As a result, the crank 8 makes the apparatus 2 easier for the operator to handle.

In order to facilitate the operator's work, the installation 1 may include a co-manipulation system for the apparatus 6, which system is connected to the proximal end of the apparatus 6. By way of example, the co-manipulation system may be a robot, an articulated arm, . . . .

In this example, the probe 9 is an ultrasound probe and it is therefore designed to be inserted in the patient's rectum. Naturally, this application is not limiting, and the holder device 2 can be used with other types of imaging, i.e. with other types of probe. The installation 1 preferably includes a needle-guide 10 secured to the apparatus 6. More precisely, the needle-guide 10 is carried by the probe 9. The needle-guide 10 is thus arranged on the apparatus 6 downstream from the crank 8 (going from the support 7 the probe 9).

Preferably, the needle-guide 10 is slidably mounted on the probe 9 so as to enable its position relative to the patient to be modified.

The needle-guide 10 also includes a grid that extends, in this example, in a plane that is substantially normal to the axis Z. The grid includes orifices 11 (only some of which are referenced in the figures), with all the orifices being parallel to one another and to the axis Z. In the grid, the various orifices 11 are arranged in columns parallel to an axis X that is orthogonal to the axis Z, and they are arranged in rows parallel to an axis Y that is orthogonal to the axis X and to the axis Z.

The needle 5 can thus be inserted in the needle-guide 10 at different heights (up the axis X) thereby serving to define the height at which the needle 5 penetrates into the perineum, and also at different widths across the needle-guide 10 (along the axis Y) thereby serving to define the distance at which the needle 5 penetrates into the perineum. It is thus possible to guide the needle 5 while having two degrees of freedom over the zone where the needle 5 is inserted into the perineum.

The grid itself preferably includes graphical marks (graduations, alphanumeric characters, . . . ) for identifying the various columns of orifices in the grid, and other graphical marks for identifying the various rows of orifices in the grid. Optionally, the grid includes letters for identifying each column and numerals for identifying each row.

There follows a description of the holder device 2.

The holder device 2 firstly includes a base 12 that is carried by the apparatus 6. More precisely, the base 12 is carried by the support 7. The base 12 is thus situated upstream from the crank 8.

The base 12 includes a strip 13 extending substantially over the entire length of the support 7 parallel to the axis Z.

Furthermore, the base 12 includes means for fastening it temporarily to the apparatus 6, such as snap fastener means. The base 12 preferably includes a first pair of arms 14 and a second pair of arms 15, the two pairs of arms being arranged at opposite longitudinal ends of the strip 13 for snap fastening the base 12 onto the support 7.

The base 12 is thus carried by the support 7 directly, without any intermediate element.

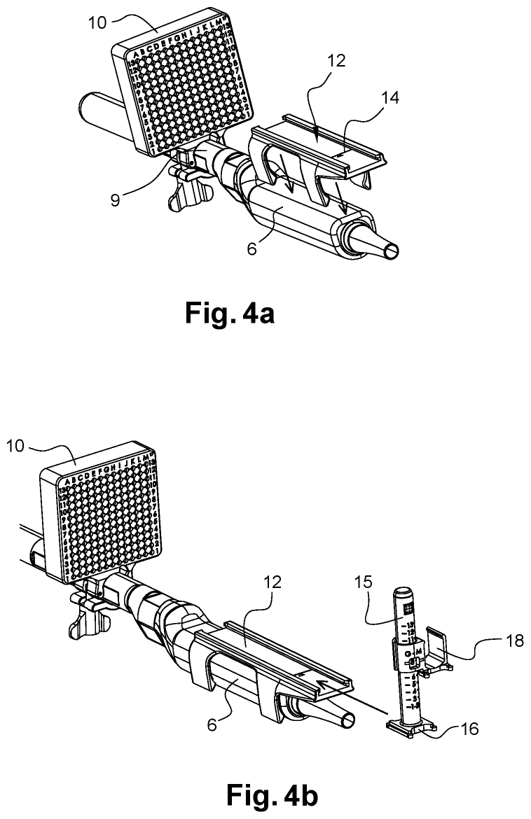

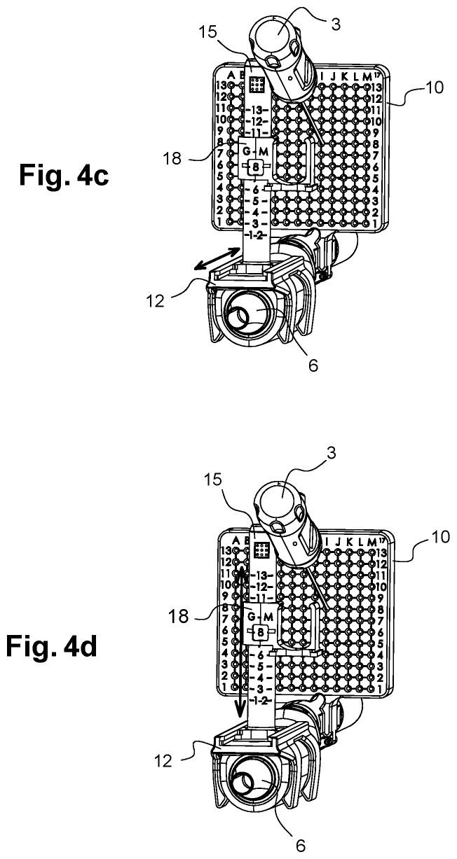

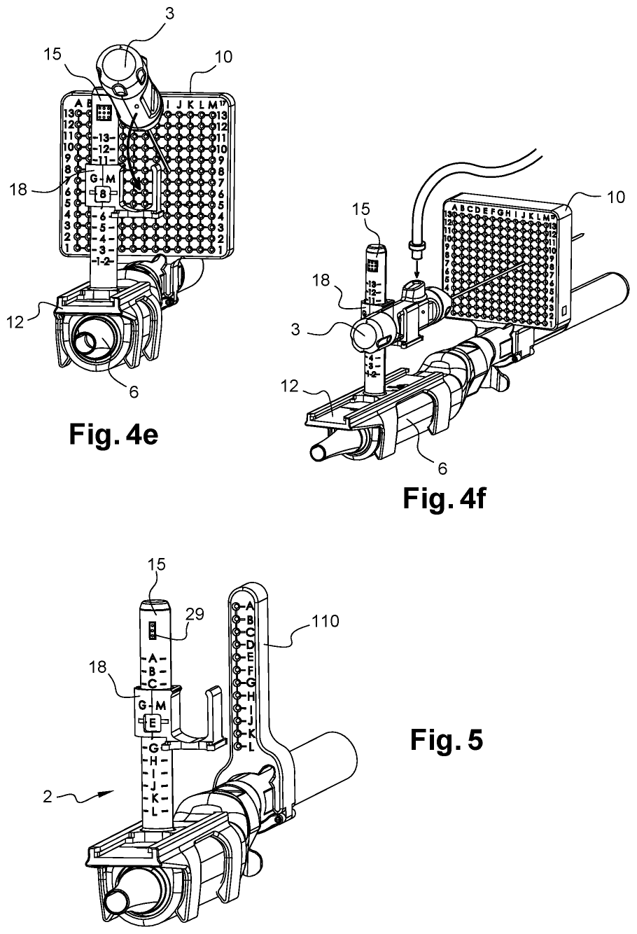

Preferably (and as is more clearly visible in FIG. 4a), the base 12 includes a position-marker sign 14 that enables the operator to place the base 12 correctly on the support 7 more quickly. By way of example, the position-marker sign 14 is a graphics element (a line, an arrow, . . . ) symbolizing one of the longitudinal ends of the support 7, and the operator arranges the base 12 in such a manner that the position-marker sign 14 is substantially in register with said longitudinal end.

The holder device 2 also includes a carrier structure, which in this example comprises a column 15 movably mounted on the base 12 so as to be capable of moving along the base 12 between a first position and a second position. In this example, the first position is said to be a “working” position, i.e. a position in which the column 15 is level with the applicator 3, and more exactly with its handle 4. In this example, the second position is said to be a “maneuvering” position, i.e. a position in which the column 15 is spaced apart from the applicator 3.

In this example, the column 15 is circular in section (except at its bottom end).

Optionally, the column 15 is mounted to slide on the base 12 between these two positions. For this purpose, the bottom end of the column 15 is shaped as a stand 16 suitable for sliding in a corresponding pair of rails 17 of the base 12. The column 15 is thus carried by the base 12 directly, without any intermediate element.

Consequently, when the column 15 is mounted on the base 12, it extends substantially normally to the base 12. In the present example, the column 15 thus extends along an axis C that, in operation, is parallel to the axis X, with the column then sliding parallel to the axis Z on the support 7.

More precisely, the rails 17 extend longitudinally parallel to the axis Z on a top face of the strip 13 so as to run along both sides of said face.

Preferably, the rails 17 extend to at least one of the longitudinal ends of the strip 13. In the present example, the rails 17 extend to both ends of the strip 13 so as to terminate at those ends. It is thus possible to separate the column 15 temporarily from the strip 13 by causing the column 15 to slide to one or the other of the longitudinal ends of the strip 13.

This makes the holder device 2 easier to assemble and to manipulate.

It is then preferable for the stand 16 to be shaped in such a manner that the column 15 can be inserted into the base 12 in only one possible orientation relative to the base 12 or else in only two possible orientations. For example, the stand 16 is not in the shape of a square but rather in the shape of a rectangle so that the stand 16 cannot be inserted with its two long sides crossing the rails 17 and/or it is shaped as a square that presents tongues suitable for co-operating with the rails 17 on only two of its sides.

Preferably, the carrier structure includes a ring 18 that is removably mounted on the column 15. The ring 18 is carried by the column 15 directly, without any intermediate element.

In this example, the ring 18 is slidably mounted on the column 15.

In preferred manner, the ring 18 is also arranged on the column 15 in such a manner as to be capable of pivoting about the axis C when the holder device 2 is in place on the apparatus 6.

In this example, the ring 18 can pivot through 360° around the column 15 about the axis C.

Typically, the ring 18 includes a tube 19 for attaching it to the column 15. The tube 19 extends along a longitudinal direction and it presents a central office that is coaxial about said direction, with the orifice passing right through the tube 19. When the tube 19 is mounted on the column 15, the longitudinal direction of the tube coincides with the general direction of the column 15, i.e. the axis C. The orifice presents a section that is similar to the section of the column 15 so as to allow the ring 18 to slide along and to turn about the column 15.

The ring 18 also has fastener means for temporarily fastening the applicator 3 to the ring 18, such as snap fastener means 20.

The snap fastener means 20 are carried by the tube 19 so as to extend beside the tube 19. In service, the snap fastener means 20 are thus arranged beside the column 15 and not on the column 15.

The snap fastener means 20 preferably comprise a plate from which two arms extend laterally for snap fastening the applicator 3 to the ring 18. The ring 3 is thus carried by the column 18 directly, without any intermediate element.

In this example, one of the arms is fastened to the tube 19. Preferably, the first arm is fastened to the tube 19 in such a manner that, when the ring 18 is in place on the column 15, the second arm extends substantially parallel to the axis C, and is spaced apart from the column 15.

Optionally, the tube 19 and the snap fastener means 20 comprise a single piece.

Preferably, the ring 18 itself includes graphical marks associated with the graphical marks of the columns of the needle-guide 10 the purpose of enabling the holder device 2 to be properly oriented relative to said needle-guide columns 10.

In the present example, and as can be seen more clearly in FIGS. 2 and 3, the ring 18 has a first graphical mark 21 on a first angular sector of the tube 19, and a second graphical mark 22 on a second angular sector of the tube 19 that is opposite from the first angular sector.

The first graphical mark 21 refers to the first half of the graphical marks of the columns (specifically indicating the first letter and the last letter of the first half), and the second graphical mark 22 refers to the second half of the graphical marks of the columns (specifically indicating the first letter and the second letter of the second half).

As a result, once the holder device 2 is in place in the installation 1, and depending on whether the ring 18 is placed so that the operator sees the first graphical mark 21 or the second graphical mark 22, the ring 18 might need to be turned through 360° , with the snap fastener means 20 thus being likewise turned through 360° . The holder device 2 is thus arranged in such a manner that the snap fastener means 20 are located either on the right or else on the left of the column 15 depending on whether it is the first graphical mark 21 or the second graphical mark 22 that faces the operator (naturally when the operator is located in the usual service position holding the support 7 and looking at the needle-carrier 10). Naturally, the snap fastener means 20 of the ring 18 are then shaped in such a manner as to be capable of receiving the applicator 3 regardless of whether the snap fastener means 20 are on the left or on the right of the column 15.

In service, and depending on the orientation of the ring 18 relative to the column 15, the applicator 3 is thus situated on the right or on the left of the column 15.

The holder device 2 thus serves to facilitate the task of the operator by carrying the applicator 3 and also by aiding the operator to position the holder device 2 relative to the applicator 3 in relatively accurate manner.

Preferably, the tube 19 is provided with two windows 23 and 24, through which it is possible to see the column 15, the two windows being arranged in opposite angular sectors of the tube 19. A respective one of the graphical marks 21 and 22 of the ring 18 is arranged above each of the windows 23 and 24.

The column 15 also has graphical marks along its axis C in association with the marks of the rows of the needle-guide 10. In this example, the graphical marks comprise a succession of numbers.

As a result, when the tube 19 is moved along the column 15, different graphical marks of the column 15 can be seen through one of the windows 23 or 24.

In the present example, the column 15 has as many graphical marks regularly distributed along its height as there are rows in the needle-guide 10, each graphical mark corresponding to a respective one of the rows of the needle-guide 10, with the exception of the bottom two rows, which are grouped together with a single graphical mark.

Optionally, the tube 19 includes at least one centering symbol 25, 26 associated with each window so that the operator places the tube 19 in such a manner that the target graphical mark 15 is properly placed at the center of the window 23 or 24 in question. In this example, each window 23 and 24 is associated with two dashes forming parts of the same horizontal middle line crossing the windows 23 or 24, the dashes being arranged on either side of the corresponding window 23 or 24. Other centering symbols could be used, such as a vertical dash, one or more arrows, one or more stars, . . . .

Preferably, each graphical mark of the column 15 is also associated with at least one centering symbol 27 (with only some of them being referenced in the drawings) so as to further facilitate centering the windows 23 or 24 under consideration on the target graphical mark of the column 15. Typically each number of the column 15 lies between two dashes belonging to the same horizontal row. It is preferable for the windows 23 and 24 to be selected to have dimensions that are large enough to ensure that at least a portion of the centering symbols 27 of the target mark of the column 15 can be seen through the window 23 or 24 in question.

Thus, the operator finds it easier to center the window 23 or 24 on the target graphical mark.

The holder device 2 thus serves to facilitate the task of the operator by carrying the applicator 3 and also by aiding the operator to position the holder device 2 accurately at the right height relative to the applicator 3.

With reference to FIGS. 4a to 4f, there follows a description of the way in which the holder device 2 is arranged in the installation 1.

With reference to FIG. 4a, the base 12 is snap fastened on the support 7, and then the probe 9 is inserted in the rectum in a particular target orientation and position for the purpose of treatment.

With reference to FIG. 4b, the column 15 is then mounted on the base 12 by inserting the stand 16 between the rails 17. The ring 18 is also put into position on the column 15.

With reference to FIG. 4c, the operator then positions the applicator 3 in the target orientation and position for the treatment while being helped in particular by the needle-guide.

Once the needle 5 is in position, it becomes necessary to hold the applicator 3 in place. For this purpose, the operator audience the ring 18 on the right-hand side if the applicator 3 is in the right-hand portion of the needle-guide 10, or on the left-hand side if the applicator 3 is in the left-hand portion of the needle-guide 10. It can clearly be seen how the graphical marks of the holder device 2 are advantageous in association with the graphical marks of the needle-guide 10 for facilitating the operator's work.

Thereafter, the operator slides the column 15 along the base 12 up to the handle of the applicator 3.

With reference to FIG. 4d, the operator then adjusts the height of the ring 18 so as to come closer to the applicator 3. Once again, it can be seen how the graphical marks of the holder device 2 are advantageous.

With reference to FIG. 4e, the operator then snap fastens the handle 4 in the ring 18. The applicator 3 is thus held in place by the holder device 2.

With reference to FIG. 4f, the operator connects the applicator 3 to a sterile cable in order to begin the treatment.

The holder device 2 as described above serves particularly, but not exclusively:

to keep the position of the applicator 3 stationary and to do so over the minutes or the tens of minutes that the treatment lasts;

to ensure that the depth position of the needle 5 is accurately controlled, with the holder device 2 preventing any involuntary sliding of the applicator 3 parallel to the axis Z;

also to be able to adjust accurately the depth to which the needle 5 is inserted into the prostate because of the possibility of moving the applicator 3 along the axis Z, the operator thus being capable of controlling the depth of insertion of the needle 5 (along the axis Z) by gently advancing the applicator 3 little by little (indeed millimeter by millimeter) along the axis Z until reaching an intended target;

to leave the operator the possibility of nevertheless having access to the entire needle-guide 10 and thus to the organ, in particular because the holder device 2 is mounted subsequently on the support 6;

to be compact, thus limiting any risk of hindering the operator during the treatment;

to be capable of being put into place and also of being removed both simply and quickly; and

to be simple in structure, thus enabling the device to be machined easily.

It should be observed that because of its two freedoms to move in translation and its freedom to move in rotation, the holder device 2 makes it possible to come closer to the applicator 3.

In the present example, advantage can usefully be taken of the flexibility of the needle 5 to allow the ring 18 to be oriented on the left-hand side or on the right-hand side only of the column 15 (as contrasted to being free to move in full parallel to the axis Y). As mentioned below, it is nevertheless possible to make provision for the holder device 2 to allow the ring 18 to move along the axis Y in order to be able to come even more accurately close to the applicator 3.

Furthermore, the simple structure of the device enables the device to be constructed very simply, e.g. by injection molding or by three-dimensional printing. In the present example, each of the base 12, the column 15, and the ring 18 is injection molded.

With reference to FIG. 5, there follows a description of a second embodiment.

Unlike the first embodiment, the needle-guide 110 is not in the form of a grid, but is in the form of a post. Consequently, the needle-guide 110 has only one column of orifices extending along the axis X.

Under such circumstances, the holder device can then be of a shape that is different from its shape in the first embodiment, having a ring that slides along the column but without pivoting about the column.

Preferably, it is nevertheless preferable to use the same holder device 2 as in the first embodiment.

Advantageously, when the needle-guide 110 does not use the same graphical marks as the needle-guide 10 of the first embodiment, the column 15 is shaped to present a first succession of graphical marks corresponding to the first needle-guide 10 and a second succession of graphical marks corresponding to the second needle-guide 110, the two successions of graphical marks being arranged on opposite angular sectors of the column 15 in register with respective ones of the two sides of the stand 16 that enable it to be inserted between the rails 17.

As a result, depending on whether it is a first side or a second side of the stand 16 of the column 15 that is inserted between the rails 17, the operator does not see the same succession of graphical marks.

Consequently, a single holder device can be used with two different needle-guides.

Optionally, the column 15 has a first direction-marker element 28 for the first succession of graphical marks and a second direction-marker element 29 for the second succession of graphical marks so that the operator can distinguish more easily between the two successions of graphical marks.

By way of example, the first direction-marker element 28 is a diagrammatic representation of a grid, and the second direction-marker element 29 is a diagrammatic representation of a post, so that the operator can easily associate the side of the column 15 that is associated with the proper needle-guide.

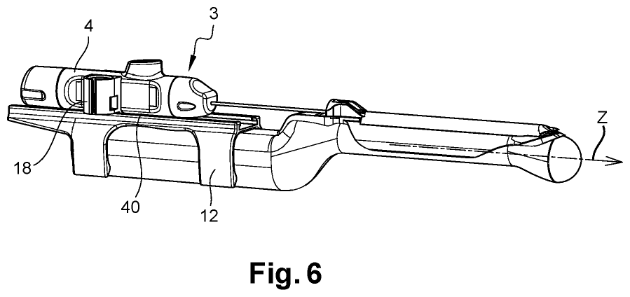

With reference to FIG. 6, there follows a description of a third embodiment.

Unlike the first embodiment, the needle-guide 210 is not in the form of a grid, but is in the form of a tunnel. Consequently, the needle-guide 210 has only one orifice.

Under such circumstances, the holder device can then be of a shape that is different from its shape in the first embodiment, such that the carrier structure does not have a column, but only a ring that is movably mounted on the base.

In the present example, the base 12 is identical to the bases of the other embodiments.

Preferably, the ring 18 is identical to the rings of the other embodiments.

Thus, in order to enable the ring 18 to slide on the base 12 between the working position in which the ring 18 is level with the applicator 3, and more exactly with its handle 4, and a maneuvering position in which the ring 18 is moved away from the applicator 3, the bottom end of the ring 18 is shaped as a stand 40 (as can be seen more clearly in FIGS. 4b and 5) that is suitable for sliding between the corresponding pair of rails 17 of the base 12. The ring 18 is thus carried by the base 12 directly, without any intermediate element.

In this example, the stand 40 is arranged at the bottom portion of the snap fastener means 20, and more particularly of its plate.

It does not matter whether the stand 40 is shaped in such a manner that the ring 18 can be inserted into the base 12 in only one possible orientation relative to the base 12 or in two possible orientations. For example, the stand 40 is not in the shape of a square but rather a rectangle so that the stand 40 cannot be inserted with its two long sides crossing the rails 17 and/or it is shaped as a square that presents tongues suitable for cooperating with the rails 17 on only two of its sides.

Advantageously, by removing the column 15 from the carrier structure and by mounting the ring 18 directly on the base 12, a single holder device can thus be used with three different types of needle-guide.

Naturally, the invention is not limited to the embodiments described, and variant embodiments may be provided without going beyond the ambit of the invention as defined by the claims.

In particular, the holder device can be used for applications other than those described. Specifically, the holder device could be used on its own and not in association with a needle-guide.

Furthermore, the installation could have some other number of instruments and/or could include instruments that are different from those mentioned as a function of the target applications. Depending on the application in which the holder device is to be used, the instrument could thus be changed. The instrument could be elongate or of some other shape. The instrument could thus be a biopsy instrument, a treatment instrument, a surgical instrument, a needle, an applicator, a fiber, . . . . If the installation includes at least two instruments, the device could thus include at least two cryotherapy applicators, at least two laser fibers, . . . . There could then be two holder devices, one for each instrument, or else there could be a single holder device carrying both instruments.

The holder device could be carried directly by the medical imaging probe.

Although above, the carrier structure is movable on the base with only a single degree of freedom to move in translation, the carrier structure could be mounted on the base with some other degree of freedom to move, such as one or more degrees of freedom to move in translation and/or one or more degrees of freedom to move in rotation.

The carrier structure could have a shape other than that described. For example, the carrier structure could have a column presenting a section (other than in its stand for attaching to the base) that is oval, square, rectangular, . . . . The carrier structure need not include a column. In the same manner, the carrier structure need not include a ring. The ring could be mounted on the column in some other way. For example, the ring could be mounted only to pivot about the general direction of the carrier column, or it could be mounted only to slide along the column. The ring could be connected to the column by a mechanism other than that described. For example, the ring could be connected to the column via a ball joint, or else via two pivot joints, or else via two siding joints. It is thus possible to have a ring that is mounted on the column to pivot about the axis of the column and also about an axis parallel to the axis X. It is thus possible to have a ring that is mounted on the column to move in translation along the axis of the column and also along an axis parallel to the axis X. The ring could have some number of windows other than that described, and for example it could have only a single window.

The carrier structure could be shaped in such a manner that all of its elements are stationary relative to one another.

It would thus be possible to have a holder device comprising a carrier structure that is mounted to be movable in translation only on the base along a single axis in such a manner that once it is held on the carrier structure, the instrument can be moved by a voluntary movement of the operator only along said axis. This makes it possible to control at least the depth to which the instrument is positioned relative to the patient. Such a device is shown in FIG. 6.

In the event of one element turning relative to another within the holder device, it may be capable of turning through a full turn (i.e. 360°) or through only part of a turn.

The various steps for mounting the holder device on the apparatus could be different from those described above. In particular, it would be possible to begin by orienting the holder device roughly relative to the instrument, and then progressively to refine the positioning of the holder device relative to the instrument. Also, although above, when the instrument is attached to the holder device, it is already in place, the instrument could be only partially in place, or not in place at all. The holder device can then help in manipulating the instrument by virtue of the various relative orientations made possible by moving the elements of the holder device.

Although above the various elements of the holder device are movable manually relative to one another, one, several, or all of the movements could be motor-driven.

Although above, friction on its own between the various elements of the holder device suffices to keep said elements in place relative to one another, the holder device could include temporary fastener means for fastening together at least two elements of the holder device (e.g. via pegs, screws, clamps, snap fastening, . . . ).

In the same manner, although the fastener means described are snap fastener means, the fastener means could be different (e.g. pegs, screws, clamps, . . . ).

1. A holder device for holding a medical instrument, the device comprising:
a base (12) that is to be carried by an apparatus (6) comprising a medical imaging probe (9) associated with the medical instrument (3); and
a carrier structure for carrying the medical instrument and movably mounted on the base so as to be capable of moving on the base between a first position and a second position.

2. A device according to claim 1, suitable for holding a medical instrument in the form of an applicator comprising a handle (4) from which there extends a treatment needle (5), the carrier structure including, for this purpose, a ring for carrying the handle of the medical instrument.

3. A device according to claim 2, wherein the ring is not carried by a needle-carrier of the device.

4. A device according to claim 1, wherein the carrier structure is slidably mounted on the base (12).

5. A device according to claim 1, wherein the carrier structure comprises a column (15) movably mounted on the base and a ring (18) movably mounted on the column.

6. A device according to claim 5, wherein the ring (18) is slidably mounted on the column (15).

7. A device according to claim 5, wherein the ring (18) is pivotally mounted on the column.

8. A device according to claim 2, wherein the ring (18) includes means for fastening the instrument temporarily to the device.

9. A device according to claim 5, wherein the ring (18) includes at least one window (23, 24) for viewing the column.

10. A device according to claim 5, wherein the ring (18) includes at least one position-marker symbol for identifying the orientation of the ring relative to the column (15).

11. A device according to claim 5, wherein the column (15) includes at least one succession of graphical marks.

12. A device according to claim 5, wherein the ring (18) is shaped to be capable of being movably mounted on the base (12) without the column (15).

13. A device according to claim 1, wherein the carrier structure can be mounted on the base (12) in at least two different engagement directions.

14. A device according to claim 13, wherein the carrier structure includes a direction-marker element for each possible direction for engaging the carrier structure on the base (12).

15. A device according to claim 1, wherein the base (12) includes at least one position-marker sign for symbolizing where the base is to be mounted in service on the apparatus.

16. An installation comprising both a device according to claim 1, and a needle-guide (10; 110; 210).

17. An installation according to claim 16, wherein the needle-guide is a grid (10).

18. An installation according to claim 16, further comprising the apparatus that includes an ultrasound probe.

19. An installation according to claim 16, further comprising the medical instrument that is not a needle-guide.
